(19) United States
(12) Patent Application Publication
(10) Pub. No.: US 2012/0046589 A1
(43) Pub. Date: Feb. 23, 2012

(54) WOUND DRESSING COMPRISING FOAM AND OINTMENT BASE FOR NEGATIVE PRESSURE THERAPY

(76) Inventors: Axel Eckstein, Heidenheim (DE); Pierre Croizat, Herbrechtingen (DE)

(21) Appl. No.: 13/213,233

(22) Filed: Aug. 19, 2011

Related U.S. Application Data
(60) Provisional application No. 61/375,090, filed on Aug. 19, 2010.

(51) Int. Cl. A61L 15/44; B05D 3/12; A61L 15/22

(52) U.S. Cl. 602/46; 602/48; 604/319; 427/2.31

(57) ABSTRACT

The invention relates to a device for negative pressure wound therapy comprising (a) a cover material for air-tight sealing of the wound space; (b) as applicable, a means for the connection of a negative pressure source; and (c) a wound dressing comprising (c1) an open-cell foam soaked with (c2) an ointment base, as well as a procedure for the manufacture of said wound dressing. The invention further relates to the use of an open-cell foam soaked with an ointment base for use as a wound dressing in negative pressure wound therapy.

The invention relates to a device for negative pressure wound therapy comprising (a) a cover material for air-tight sealing of a wound space; (b) as applicable, a means for the connection of a negative pressure source; and (c) a wound dressing comprising (c1) an open-cell foam soaked with (c2) an ointment base, as well as a procedure for the manufacture of the said wound dressing. The invention further relates to the use of an open-cell foam soaked with an ointment base for use as a wound dressing in negative pressure wound therapy.

A wound is defined as the separation of the coherence of tissues of the outer body of humans or animals. It can result in a loss of substance.

Devices for the negative pressure wound therapy are known in the prior art.

For example, WO 1993/009727 Al describes a device to promote healing of the wound by the application of a negative pressure on the skin area which is wounded and the area surrounding the wound. The device according to WO 1993/009727 A1 comprises a negative pressure device to generate the negative pressure, an air-tight cover of the wound which is functionally connected to the negative pressure device, as well as a wound dressing for positioning on the wound inside the air-tight cover.

Devices for negative pressure wound therapy are commercially available, for example the V.A.C.® device from the company KCI. Commercially available devices often use a wound dressing which contains an open-cell polymer foam such as polyvinyl alcohol (PVA) or polyurethane (PU).

The commercially available foam dressings are compressed to a different degree, depending on the negative pressure applied. This can cause a constriction of the passages necessary for the removal of the wound exudate. Adhesion of the foam with the wound can also occur. Newly formed tissue can grow into the wound. This problem is a familiar complication in the negative pressure therapy of wounds (FDA complaint data base). In order to solve this problem, additional wound contact layers are often introduced between the foam and the wound, for example a film (see, for example, WO2001/85248). However, these additional wound contact layers can reduce the passage of wound exudate.

When the wound dressing is to be changed, elaborate measures have to be taken to remove adhered foam, for example by rinsing with Ringer's solution. Tissue which has grown into the foam can lead to a tissue traumatization when the wound dressing is removed and thus delay the healing process.

When conventional wound dressings are used, particles of foam can also enter the wound. This problem is aggravated if the wound dressing is cut to the size of the wound before being applied, as this results in the generation of loose foam particles at the cut edges. It has also been shown that the usual polymer foams can also get partially stuck to the cover film. The adhesion of the wound dressing with the cover film is a particular disadvantage when the wound dressing is to be removed.

It is the object of the present invention to further improve negative pressure wound therapy and to overcome the disadvantages of the prior art. The present invention provides devices and methods for negative pressure wound therapy, with which a therapy can be carried out as effectively and comfortably as possible.

In particular, the present invention is intended to allow a negative pressure wound therapy which enables the patient to change the wound dressing at intervals of, for example, up to 3 days. During the interval the wound dressing should be subjectively perceived by the patient as pleasant. There should be no irritation by pressure or reddening of the skin. When the wound dressing is changed after a period of, for example, up to 3 days, there should be as little unpleasant odor as possible. The germ density in the exchanged wound dressing should be low.

The object is to provide a wound dressing which avoids, as far as possible, adhesion with the cover film. The suction force should not be reduced depending on the distance to the port.

Unexpectedly, the objects could be solved by the use of a wound dressing which contains an open-cell foam soaked with an ointment base. Surprisingly, it was also found that a device for negative pressure therapy comprising at least a wound dressing soaked with an ointment base is especially suitable for an advantageous, i.e. very effective and very gentle treatment of wounds.

It was found that the objects could be solved particularly advantageously if a special foam and/or a special ointment base are used and/or if the type of foam and the type and quantity of the ointment base are mutually coordinated.

The object of a first aspect of the invention is, therefore, a device for negative pressure wound therapy comprising

(a) a cover material for air-tight sealing of the wound space;

(b) as applicable, a means for the connection of a negative pressure source; and

(c) a wound dressing comprising

(c1) an open-cell foam which is

(c2) soaked with an ointment base, which has a dropping point of 20 to 80° C.

The proportion of ointment base is preferably 10 to 95 weight per cent relative to the total weight of the wound dressing.

The object of a second aspect of the invention is, therefore, a device for negative pressure wound therapy comprising

a) a cover material for air-tight sealing of the wound space;

(b) as applicable, a means for the connection of a negative pressure source; and

(c) a wound dressing comprising

(c1) an open-cell foam which is

(c2) soaked with an ointment base on a triglyceride basis.

The object of a third aspect of the invention is, therefore, a device for negative pressure wound therapy comprising

(a) a cover material for air-tight sealing of the wound space;

(b) as applicable, a means for the connection of a negative pressure source; and

(c) a wound dressing comprising

(c1) a special open-cell foam (see the explanations below for component (c1)), whereby the foam (c1) has, in particular, an air permeability of 1,000 to 8.000 l/(m²sec), measured in accordance with DIN EN ISO 9237, and is soaked with (c2) an ointment base. The proportion of ointment base is preferably 10 to 95 weight per cent relative to the total weight of the wound dressing.

The invention also comprises any combinations of the cited aspects. A further object of the invention is the use of an open-cell foam which is soaked with an ointment base, preferably an ointment base with a dropping point of 20 to 80 ° C., in particular with a triglyceride ointment base as a wound dressing for or in negative pressure wound therapy.

A further object of the invention is a method for the manufacture of a wound dressing, in particular a wound dressing used in negative pressure therapy, comprising the steps of

(I) heating an ointment base above the dropping point,

(II) introducing an open-cell foam into the heated ointment base, so that the foam is soaked with the ointment base,

(III) as applicable, temporarily compressing the foam, preferably to at least 50% and up to a maximum of 90% of its original volume in order to ensure that the internal surface of the foam is fully soaked with the ointment base, and

(IV) as applicable, removing the surplus ointment base, preferably by squeezing the foam. In addition to this, wound dressings which can be obtained using the method in accordance with the present invention, are an object of the invention.

The new device in accordance with the present invention and the use of the wound dressing in accordance with the present invention are characterized by several unexpected advantages.

By soaking the foam with the ointment base it was possible to advantageously reduce the number of unwanted particles entering the wound.

The use of the wound dressing in accordance with the present invention improved the atraumatic characteristics so that a negative pressure therapy was possible without additional wound contact layers.

Soaking the foam with the ointment base reduced the hardness of the foam as perceived subjectively by the patient during the negative pressure therapy. The foam felt more pleasant and the patient compliance (observance of the therapy instructions by the patient) was increased.

It was also shown that, despite the use of the ointment base, the suction force is not undesirably reduced depending on the distance from the port. Surprisingly, with the wound dressing in accordance with the present invention it is possible to achieve both sufficient drainage of wound exudate as well as an effect which supports the healing of the wound, particularly if an ointment base is used which contains triglycerides and, as applicable, diglycerides. This could be due to the activity of the endogenous lipoprotein lipase present in the wound. The fatty acids released from the triglycerides in the ointment base appear to have a positive effect on the healing of the wound.

An effect which supports the healing of the wound can be achieved with the wound dressing in accordance with the present invention in particular if the wound dressing provides a sufficient amount of ointment to the wound. Too much ointment base can, however, block the pores and thus obstruct the passage of wound exudate. The proportion of ointment base is preferably 10 to 95 weight per cent, more preferably 30 to 92 weight per cent, even more preferably 45 to 90 weight per cent, especially preferably 55 to 88 weight per cent, in particular 65 to 85 weight per cent relative to the total weight of the wound dressing.

The method of manufacture in accordance with the present invention is technically simpler and can also be used with medical foams purchased as a finished product. The wound dressing is uniformly soaked, which reduces the release of particles in all areas of the foam when the foam is cut. In contrast to the methods known from the prior art, in which the impregnation agent is added before the foam is set or synthesized (see EP 0 335 669), there are no unwanted interactions between the ointment base and the foam on setting or synthesis.

A further advantage of the wound dressing in accordance with the present invention is that an adhesion and/or a growing together of the wound with the wound dressing even over a period of, for example, up to 3 days can be largely avoided. The traumatization of the wound when the wound dressing is changed can be avoided. This increases the efficacy of the wound therapy, thus allowing the change of the patient's wound dressing at intervals of, for example, up to three clays. During the interval of 3 days, the wound dressing in accordance with the present invention was perceived as pleasant by the patient. Irritation by pressure and reddening of the skin were generally avoided. When changing the wound dressing after a period of 3 days, there were few unpleasant odors. The germ density in the changed wound dressing was unexpectedly low.

The components (a) to (c) of the device in accordance with the present invention are described in the following.

The device in accordance with the present invention comprises a cover material (a) for air-tight sealing of the wound space. The wound space is regarded as the wound and. as applicable, the area surrounding the wound. “Air-tight sealing” does not mean that there is no exchange of gas between the wound space and its surroundings. Rather, “air-tight sealing” in this context means that, taking into account the vacuum pump used, the negative pressure necessary for the negative pressure wound therapy can be maintained. This means that cover materials can also be used which have a slight degree of gas permeability as long as the negative pressure necessary for the negative pressure therapy can be maintained.

In a preferred embodiment of the invention, the cover material for the air-tight sealing of the wound comprises a water-insoluble polymer, or a metal foil. The cover material preferably has a thickness of 10 μm to 10,000 μm, in particular from 25 μm to 100 μm.

In a preferred embodiment of the invention, the cover material (a) is a water-insoluble polymer. Preferably the water-insoluble polymer has a solubility of 10 mg/ml or less, more preferably of 1 mg/ml or less, particularly from 0.0001 to 1 mg/ml (determined in accordance with the column elution method pursuant to EU Directive RL67-548-EEC, Annex V, Chapter A6). Examples include polyurethane, polyester, polypropylene, polyethylene, polyarnide or polyvinyl chloride, polyorganosiloxane (silicone), or a mixture thereof. The cited polymers are preferably present in non-cellular form.

It has been demonstrated that the objects explained at the beginning can be solved in a particularly advantageous manner using a cover material with a specific water vapor permeability. In a preferred embodiment, the cover material thus has a water vapor permeability of 100 to 2,500 g/m²×24h, more preferably from 500 to 2,000 g/m²×24h, and even more preferably from 800 to 1,600 g/m²×24h, in particular from 1,050 to 1,450 g/m²×24h, determined in accordance with DIN EN 13726-2 at 23° C. and 85% relative humidity. In particular, the combination of a cover film (a) having the aforementioned water vapor permeability with an open-cell foam (c 1) having the physical properties described below is particularly advantageous.

In a preferred embodiment, the cover material and the means for the connection of a negative pressure source are supplied already connected and ready to use. It is most especially preferred that this embodiment comprises a film of one or more water-insoluble polymers with a self-adhesive edge, as this arrangement considerably facilitates the application of the dressing.

The device in accordance with the present invention for negative pressure therapy comprises a means (b) for the connection of a negative pressure source, i.e. a means for the generation of a negative pressure in the wound space. In a preferred embodiment, this is a means (b) for the functional connection of the wound space with a negative pressure source outside of the cover material so that a negative pressure can be generated in the wound space and fluids can be sucked out of the wound space.

The expression “negative pressure in the wound space” in the context of the invention describes an air pressure which is lower inside the wound dressing compared to the atmospheric pressure. “Within the wound dressing” refers to the cavity formed between the cover material and the wound.

The pressure difference between the air pressure inside the wound dressing and the atmospheric pressure is stated in the context of the invention in mm Hg (millimeters of mercury), as this is the convention in negative pressure therapy. 1 mm Hg corresponds to one torr or 133.322 Pa (Pascal). In the context of the invention, the negative pressure. i.e. the pressure difference between the pressure inside the wound dressing and the atmospheric pressure, is stated as a positive numerical value in mm Hg.

In one embodiment of the invention, the negative pressure is at least 25 mm Hg up to a maximum of 250 mm Hg, preferably at least 50 mm Hg up to a maximum of 150 mm Hg. This negative pressure range has proved suitable for wound healing. In a preferred embodiment of the invention, the negative pressure is at least 80 mm Hg up to a maximum of 140 mm Hg, more preferably at least 120 mm Hg up to a maximum of 130 mm Hg.

The device in accordance with the present invention for negative pressure wound therapy preferably comprises, as set out above, a means (b) for connection of a negative pressure source, i.e. a means for the functional connection of the wound space with a negative pressure source outside of the cover material.

The functional connection can be generated, for example, by a connection line or by a negative pressure connector. Negative pressure connectors are known to those skilled in the art as “ports”. In one embodiment, the means (b) is a connection line, preferably a tube, in particular a silicone drainage tube. The connection line can be ducted through the cover material. Alternatively, the at least one connection line can be led under the edge of the cover material. In both cases the penetration point must be sealed air-tight so that the desired negative pressure can be maintained in the dressing. Sealing can be achieved, for example, with an adhesive foil, an adhesive paste or an adhesive strip. The connection line can be, for example, a tube, a pipe or any other hollow body.

In a further preferred embodiment, the means (b) is a negative pressure connector (port) which can be fastened to one of the inner or outer sides of the cover material, whereby the cover material has the corresponding openings. In this embodiment it is also important to ensure air-tight sealing either of the penetration opening (inside port) or the surface of the dressing (outside port). Sealing can be achieved, for example, with an adhesive foil, an adhesive paste or an adhesive strip. It is also conceivable that the port itself has the corresponding fastening facilities, such as adhesive surfaces. Suitable negative pressure connectors are commercially available. These are typically negative pressure connectors which are fastened on the outside of the cover material. The negative pressure connector should also have a negative pressure adapter so that it can be connected to the other components of the negative pressure system.

Alongside the components (a) and, optionally, (b) described above, the device in accordance with the present invention also has a component (c). The wound dressing (c) used in the device in accordance with the present invention is described in more detail in the following. All of the explanations on the wound dressing (c), including the components (c1) and (c2), refer not only to the device in accordance with the present invention, but also to the method in accordance with the present invention for the manufacture of the wound dressing and the use in accordance with the present invention of the wound dressing in negative pressure therapy.

The wound dressing (c) comprises of an open-cell foam (c 1) and an ointment base (c2).

Foams are usually materials with cells (open, closed, or both) distributed over their whole mass. Such materials thus usually have a raw density (in accordance with DIN EN ISO 845) which is lower than the density of the basic substance.

A cell is an individual cavity formed during the manufacture of the foam which is partially or fully enclosed by the cell walls and/or cell struts.

A closed cell is usually a cell which is completely enclosed by its walls and has no connection via the gas phase with the other cells. An open cell is usually a cell which is connected with other cells via the gas phase. In the context of this application, the term open-cell means that in the foam (c1) there is at least 60% open cells, preferably at least 90% open cells, even more preferably 98% open cells, in particular essentially 100% open cells relative to the total number of cells. The open cell content of the polyurethane foam is usually determined in accordance with ASTM D 2856-87, procedure B.

The cell wall is usually taken to mean the wall enclosing the cell. The cell wall can also be referred to as the cell membrane. The cell strut is usually taken to mean the area of the cell wall which separates more than two cells. Cell struts are preferably at least 1.5 times the thickness, even more preferably at least twice the thickness of the rest of the cell wall.

The open-cell foam (c 1) can be a reticulated foam. A reticulated foam is taken to mean a foam which consists largely of cell struts. In a reticulated foam, therefore, the cell walls are largely absent. The reticulation is usually carried out in a pressure chamber, e.g. a steel chamber. When the foam is introduced to the steel chamber, the air is sucked out (preferably from 50 to 100 weight per cent, more preferably from 70 to 99 weight per cent) and replaced by a combustion gas mixture, preferably by a mixture containing hydrogen and oxygen, in particular in a molar ratio of 2:1. When the gas mixture is ignited, the cell skins are torn by the resulting heat and the pressure wave. There may also be at least a partial melting of the cell struts so that these are reinforced.

The foam (c1) preferably has a cell number (=number of pores along a straight line per cm) of 5 to 25 cm⁻¹, more preferably 7 to 18 cm⁻¹, even more preferably 8 to 15 cm⁻¹. The cell number is preferably determined by microscope.

In principle, the open-cell foam can be of any material. It should, however, fulfill certain physical requirements. It has been demonstrated that the objects set out above can be solved unexpectedly advantageously if the foam (c1) has a specific tensile strength, a specific ductile yield and/or a specific hardness. In a preferred embodiment, the foam (c1) has a tensile strength of 100 kPa to 500 kPa, more preferably 120 kPa to 300 kPa, even more preferably 140 kPa to 250 kPa, measured in accordance with DIN 53571 (test body A, pretreatment in standard climate).

Furthermore, the foam (c1) preferably has a ductile yield of 150% to 700%, more preferably from 200% to 650%, even more preferably from 240% to 400%, particularly 260% to 320%, measured in accordance with DIN 53571 (Procedure 1. test body A). In addition to this, the foam (c1) preferably has a hardness of 20 to 70 Shore A, more preferably from 30 to 60 Shore A, even more preferably from 40 to 50 Shore A, measured in accordance with DIN 53505, whereby the measurement was taken at 23° C. on a slab-like, flat and smooth test body with a thickness of 6 mm.

It has also been demonstrated that the objects set out above can be solved unexpectedly advantageously if the foam (c1) has a specific air permeability. In a preferred embodiment the foam (c1) has an air permeability of 1,000 to 8,000 l/(m²sec). more preferably from 1,500 to 6,000 l/(m²sec), even more preferably from 2,000 to 5,000 l/(m²sec), in particular from 2,300 to 4,000 l/(m²sec), measured in accordance with DIN EN ISO 9237 (20 mm test thickness, 20 cm² test area, 200 Pa differential pressure).

It has also been demonstrated that the objects set out above can be solved unexpectedly advantageously if the foam (c1) displays visco-elastic behavior. This means that the behavior of the foam (c) under strain looks like a combination of an elastic solid and a viscous fluid. The visco-elastic behavior can be characterized by a torsional vibration test in accordance with DIN 53445, Procedure A. It is preferred that the foam when determined in accordance with DIN 53445, Procedure A at 23° C. has a mechanical loss factor of 0.1 to 1.0, more preferably from 0.15 to 0.8, even more preferably from 0.2 to 0.6.

It has also been demonstrated that the objects set out above can be solved unexpectedly advantageously if the foam (c1) has a raw density between 15 and 55 kg/m³, more preferably between 20 and 45 kg/m³, even more preferably between 22 and 38 kg/m³, in particular between 23 and 35 kg/m³, measured in accordance with DIN EN ISO 845 (test body with dimensions 100 mm×100 mm×50 mm, whereby the measurement was carried out in standard climate, i.e. at 23° C. and 50% relative humidity, and the test body was conditioned to the standard climate for 24 hours before the measurement).

In a preferred embodiment the open-cell foam (c1) is selected from polyurethane foams (PUR), (preferably cross-linked) polysiloxane foams (SIL) and polyvinyl alcohol foams (PVA). Polyurethane foams are especially preferred.

Preferred embodiments of the usable polyurethane foams (c1-PUR) are explained below. The polyurethane foam is usually obtainable by reaction of a curable mixture comprising the components

(i-PUR) polyisocyanate,

(ii-PUR) compounds reactive to isocyanate, in particular polyol,

(iii-PUR) catalyst,

(iv-PUR) blowing agent, and

(v-PUR) additives, as applicable.

Generally known aliphatic, cycloaliphatic and/or, in particular, aromatic polyisocyanates can be used as isocyanates (i-PUR). For example, diphenylmethane diisocyanate (MDI), in particular, 4,4′-diphenylmethane diisocyanate (4,4′-MDI), mixtures of monomeric diphenylmethane diisocyanates and higher-nucleus homologues of diphenylmethane diisocyanate (polymeric MDI), tetramethylene diisocyanate (TMDI), hexamethylene diisocyanate (HDI), toluylene diisocyanate (TDI) or mixtures thereof can be used to produce the polyurethanes.

Preference is given to MDI, in particular 4,4′-MDI and/or HDI. The preferably used 4,4′-MDI can contain small quantities, up to around 10 weight per cent, of allophanate or uretonimine-modified polyisocyanates. Small quantities of polyphenylene polymethylene polyisocyanate (PMDI) can also be used. The total quantity of these PMDI should not exceed 5 weight per cent of the isocyanate used.

The polyisocyanate component (a) is preferably used in the form of polyisocyanate prepolymers. These polyisocyanate prepolymers are obtainable by reaction of the polyisocyanates described above (i-PUR), for example at temperatures of 30 to 100° C., preferably at around 80° C., with a substoichiometric amount of the polyols (ii-PUR) described below to form a prepolymer. The polyol-polyisocyanate ratio is selected in such a way that the NCO content of the prepolymer is 8 to 28 weight per cent, preferably 14 to 26 weight per cent, particularly preferably 17 to 23 weight per cent.

Polyols such as polyetherols and/or polyesterols are usually used as compounds reactive to isocyanates (ii-PUR).

It is also possible to use polyether polyalcohols (referred to in this application as “polyether polyols”) with an OH functionality of 1.9 to 8.0, a hydroxyl number of 50 to 1,000 mg KOH/g and, as applicable, 10 to 100% primary hydroxyl groups. These types of polyether polyols are known, commercially available and based, for example, on starter compounds which can be reacted with alkylene oxides, for example propylene oxide and/or ethylene oxide, under generally known conditions. The content of primary hydroxyl groups can be achieved by eventually reacting the polyols with ethylene oxide. To produce the open-cell foam (c) it is preferable not to use polyether polyols.

Preference is given to the use of polyester polyols in the component (ii-PUR). The polyesterols (ii-PUR) are usually produced by condensation of multifunctional alcohols, preferably diols, with 2 to 12 carbon atoms, preferably 2 to 6 carbon atoms, with multifunctional carboxylic acids with 2 to 12 carbon atoms, for example succinic acid, glutaric acid, adipic acid, phthalic acid, isophthalic acid and/or terephthalic acid and mixtures thereof. Examples of suitable di- and multi-valent alcohols include ethanediol, diethylene glycol, 1,4-butanediol, 1,5-pentanediol, and/or 1,6-hexanediol and mixtures thereof.

The reaction conditions of carboxylic acid and alcohol are usually selected in such a way that the resulting polyesterols do not have any free acid groups. The resulting polyesterols also generally have a weight average molecular weight (determined using gel permeation chromatography) of 500 to 3,000 g/mol, preferably of more than 1,000 g/mol to 2,500 g/mol. In general, the polyesterols used have an average theoretical functionality of 2.0 to 4, preferably of more than 2 to less than 3. The polyesterols used also generally have an average OH number of 20 to 200, preferably from 30 to 90.

In a preferred embodiment, the polyesterols used have a viscosity of 150 mPa.s to 600 mPa.s, preferably from 200 mPa.s to 550 mPa.s, more preferably from 220 mPa.s to 500 mPa.s, especially preferably from 250 mPa.s to 450 mPa.s and in particular from 270 mPa.s to 350 mPa.s. measured in accordance with DIN 53 015 at 75 ° C.

The compounds (ii-PUR) can be mixed with chain extenders and/or cross-linking agents. The chain extenders are mainly 2-functional alcohols with molecular weights from 60 to 499, for example ethylene glycol, propylene glycol, butanediol-1,4, pentanediol-1,5, dipropylene glycol and/or tripropylene glycol. The cross-linking agents are compounds with molecular weights from 60 to 499 and 3 or more active H atoms, preferably amines, and especially preferably alcohols, for example glycerin, trimethylol propane and/or pentaerythrite.

In a preferred embodiment the component (ii-PUR) preferably contains (or consists of 0 to 25 weight per cent, preferably 1 to 20 weight per cent, chain extenders and/or cross-linking agents and 75 to 100 weight per cent, preferably 80 to 99 weight per cent polyol(s), in particular polyester polyol(s), relative to the total weight of the component (ii-PUR).

The usual compounds can be used as catalysts (iii-PUR) to accelerate the reaction of the component (i-PUR) with the component (ii-PUR). These could include, for example. tertiary amines and/or organo-metallic compounds, in particular tin compounds. The following compounds can be used, for example, as catalysts: triethylene diamine, aminoalkyl and/or aminophenyl imidazoles and/or tin (II) salts of organic carboxylic acids. Catalysts are generally used in a quantity of 0.1 to 5 weight per cent relative to the weight of the component (ii-PUR).

Generally known chemically or physically active compounds can be used as blowing agents (iv-PUR). Water can be used preferably as a physically active blowing agent, which, when reacted with the isocyanate groups, forms carbon dioxide. Examples of physical blowing agents include (cyclo)aliphatic hydrocarbons, preferably those with 4 to 8, especially preferably 4 to 6, and in particular 5 carbon atoms, partially halogenated hydrocarbons or ethers, ketones or acetates. The amount of blowing agents used depends on the desired density of the foams. The different blowing agents can be used individually or in any mixture with each other. Special preference is given to the use of only water as a blowing agent, generally in a quantity of 0.1 to 5 weight per cent, in particular from 2.5 to 4 weight per cent relative to the weight of the component (ii-PUR). Physical blowing agents are preferably used in a quantity of <0.5 weight per cent relative to the weight of the component (ii-PUR).

The reaction takes place as applicable in the presence of (v-PUR) auxiliaries and/or additives, for example fillers, cell regulators, cell openers, surfactants, and/or stabilizers against oxidative, thermal or microbial decomposition or ageing.

To produce polyurethane foams, the components (i-PUR) and (ii-PUR) are generally made to react with each other in such quantities that the equivalence ratio of NCO groups to the sum of the reactive hydrogen atoms is 1:0.8 to 1:1.25, preferably 1:0.9 to 1:1.15. A ratio of 1:1 corresponds here to an NCO index of 100. The desired open cell content of the polyurethane foam is generally achieved by a suitable selection as recognized by those skilled in the art of the components (i-PUR) to (v-PUR). Preferably, after setting the resulting PUR foam is reticulated. For more information on this, reference is made to the explanations given above. Preferred embodiments of the useable, preferably cross-linked polysiloxane foams (c1-SIL) are explained in the following.

In accordance with a further embodiment, the open-cell foam is a polysiloxane foam. Polysiloxanes are generally taken to be synthetic polymers in which silicon atoms are linked by oxygen atoms.

In a preferred embodiment the foam (c1-SIL) is obtainable by reaction of a curable mixture comprising the following components:

(i-SIL) a polyorganosiloxane containing one or more groups with a C₂—C₁ alkenyl group, preferably containing one or more vinyl groups,

(ii-SIL) a polyorganosiloxanc containing one or more Si—H groups,

(iii-SIL) a blowing agent containing one or more OH groups, and

(iv-SIL) an organo-metallic catalyst.

In a third embodiment the open-cell foam can be a polyvinyl alcohol foam (PVA-foam). The commercially available PVA foams are, in principle, suitable for this purpose, but should preferably have the abovementioned physical properties.

It has also been demonstrated that the objects set out above can be solved unexpectedly advantageously if the foam (c1) contains silver in the form of silver ions or in the form of atomic silver. Preferably, a silver coating is applied after production of the foam (c1). Alternatively the silver can be added to the curable composition. Preferably the foam (c) contains 0.000001 to 0.1 weight per cent, more preferably 0.0001 to 0.01 weight per cent silver relative to the total weight of the foam (c1).

In a preferred embodiment of the invention, the open-cell foam has a thickness of 1 to 50 mm, in particular from 15 to 35 mm.

In a preferred embodiment, the open-cell foam (c1) is used in a dry condition and soaked with the ointment base (c2). Preferably, the foam is not, for example, soaked with an activation solution (e.g. Ringer's solution).

In principle, the ointment bases known to those skilled in the art and described in Bauer, Frömming, Führer “Lehrbuch der Pharmazeutischen Technologie”, 8^{th} edition, chapter 12.1 to 12.6 are suitable. Accordingly, an ointment base is taken to be a spreadable, semi-solid preparation which is suitable in principle for use on the skin or mucous membranes, but which (still) does not contain any pharmaceutical active ingredients. The production of ointment bases is known to those skilled in the art; reference is made to Ph. Eur. 6.0 “Semi-solid preparations for cutaneous application”.

Preference is given to the use of ointment bases (c2) which do not contain an aqueous phase. Preference is given to hydrophobic, water-absorbing and/or hydrophilic ointment bases. Hydrophobic ointment bases are preferred.

Hydrophobic ointment bases are those which essentially contain no polar constituents and thus do not actively bind water. We thus refer to a lipophilic base in which water can only be incorporated by mechanical dispersion. Examples of preferred hydrophobic ointment bases include hydrocarbon bases (e.g. vaseline or vaseline-paraffin mixtures), diglycerides, triglycerides, waxes, polyalkylsiloxanes and mixtures thereof. Preference is given in particular to triglycerides or mixtures of triglycerides and diglycerides as an ointment base (c2).

Water-absorbing ointment bases contain lipophilic substances and surfactants. Examples of water-absorbing ointment bases include W/O emulsions (e.g. lanolin) or O/W emulsions.

Hydrophilic ointment bases are preparations which are water-miscible. A preferred example is a polyethylene glycol ointment base (PEG with weight average molecular weight of usually 300 to 4,000 g/mol, preferably 1,500 to 3,000 g/mol).

Alongside the preferred ointment bases described above, the component (c2) also comprises cream bases (hydrophilic and hydrophobic cream bases), gels (hydrophobic gels, hydrophilic gels) and pastes (suspension ointment bases).

The ointment base (c2) should have a semi-solid consistency. It has proved advantageous in solving the objects cited at the beginning if the ointment base has a dropping point of 20 to 80° C., preferably from 25 to 55° C. more preferably from 30 to 50° C., even more preferably from 33 to 48° C. and in particular from 35 to 45° C. The dropping point is the temperature at which the first drop of a melting substance detaches itself from the metal nipple of a dropping point thermometer. For the experimental determination of the dropping point, reference is made to the explanations on FIG. 2 below.

Alongside the dropping point, it has proved advantageous in solving the objects cited at the beginning if the ointment base (c2) fulfills one or more of the following parameters:

Acid number from 0.001 to 2.0 mg KOH/g, determined in accordance with Ph. Eur. 6.0, 2.5.1;

iodine number from 0.001 to 3.0 g 12/100 g, determined in accordance with Ph. Eur. 6.0, 2.5.4;

peroxide number from 0.001 to 1.0 mequi O/kg, determined in accordance with Ph. Eur. 6.0, 2.5.5 A;

OH number from 1 to 100, preferably from 5 to 90 mg KOH/g, determined in accordance with Ph. Eur. 6.0, 2.5.3:

saponification number from 200 to 350 mg KOH/g, preferably from 240 to 300 mg KOH/g, determined in accordance with Ph. Eur 6.0, 2.5.6;

heavy metal content of a maximum of 10 ppm, determined in accordance with Ph. Eur. 6.0, 2.4.8.D.

Insofar as the standards and Ph. Eur. regulations cited in this application do not state otherwise, the tests are generally carried out in standard climate, i.e. at 23° C. and 50% relative humidity and at an atmospheric pressure of 1013 mbar.

In an especially preferred embodiment, triglycerides are used as an ointment base (c2).

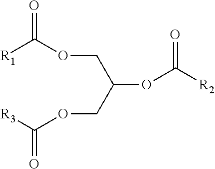

R₁, R₂ and R₃ can be the same or, preferably, different.

In a preferred embodiment, the triglyceride contains a glycerin residue and three C₆—C₂₈ acid residues, preferably C₈—C₁₈ acid residues. The acid residues can he saturated or unsaturated, preference is given to saturated fatty acids. The acid residues can, as applicable, be substituted, for example with a hydroxyl group.

In an especially preferred embodiment the acid residues of the triglycerides contain caprylic acid, capric acid, lauric acid and/or stearic acid. Preference is given here in particular to triglycerides in which the fatty acid fraction contains and in particular consists of 20 to 40 weight per cent caprylic acid, 10 to 30 weight per cent capric acid, 5 to 20 weight per cent lauric acid and 30 to 50 weight per cent stearic acid.

In an alternative, especially preferred embodiment diglycerides are used as the ointment base (c2).

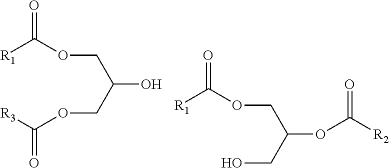

R₁ and R₂ can be the same or, preferably, different.

In a preferred embodiment, the diglyceride contains a glycerin residue and two C₄—C₂₈ acid residues, preferably C₆—C₁₈ acid residues. The acid residues can be saturated or unsaturated, preference is given to saturated fatty acids. The acid residues can, as applicable, be substituted, for example, with a hydroxyl group. It is particularly preferable that the acid residues of the isostearic acid contain stearic acid, 12-hydroxystearic acid and/or adipic acid.

In an especially preferred embodiment the ointment base (c2) contains a mixture of triglycerides and diglycerides. The ointment base (c2) especially contains 25 to 90 weight per cent, preferably 45 to 80 weight per cent triglycerides, and 10 to 75 weight per cent, preferably 20 to 55 weight per cent diglycerides.

It is also preferable that polyethylene glycol (PEG) is added to the mixture of tri- and diglycerides. In particular the mixture of tri-/diglycerides/PEG contains 1 to 30 weight per cent, preferably 5 to 20 weight per cent polyethylene glycol relative to the total weight of the mixture. Preference is given here to PEG with a weight average molecular weight of 500 to 3,000 g/mol, in particular from 1,500 to 2,500 g/mol.

An especially preferred ointment base contains: 20 to 90 weight per cent, preferably 55 to 80 weight per cent triglycerides, in particular containing fatty acid residues selected from caprylic acid, capric acid, lauric acid and stearic acid; 5 to 75 weight per cent, preferably 15 to 45 weight per cent diglycerides, in particular containing fatty acid residues selected from isostearic acid, stearic acid, 12-hydroxy-stearic acid and/or adipic acid; and

0 to 30 weight per cent, preferably 5 to 20 weight per cent polyethylene glycol with a weight average molecular weight of 500 to 3,000 g/mol.

Alongside di- and triglycerides the ointment base can also preferably contain fatty alcohols, alkoxylated fatty alcohols, fatty acids and alkoxylated fatty acids.

The ointment base (c2) preferably contains no monoglycerides.

In particular, the ointment base (c2) contains no glycerol monooleate.

In a preferred embodiment the ointment base (c2) can also contain substances with antimicrobial action. Substances with antimicrobial action can include for example, substances with amino or imino groups. Substances with antimicrobial action can also be antimicrobially active metal cations, in particular silver cations, for example a complex of 1-vinyl-2-pyrrolidones with silver cations. Other especially suitable substances with antimicrobial action further include biguanide derivatives such as chlorhexidine or polybiguanides such as polyethylene biguanide (PEB), polytetramethylene biguanide (PTMB) or polyethylene hexamethylenc biguanide (PEHMB). An especially preferred polybiguanide is polyhexamethylene biguanide (PHMB or polyhexanide). Other suitable substances with antimicrobial action are polyguanidines such as polyhexamethylene guanidine (PHMG), N-octyl-1-[10-(4-octyliminopyridine-1-yl)decyl]pyridine-4-imine (octenidine), quaternary ammonium compounds such as benzalkonium chloride or cetylpyridinium chloride, triazines such as 1-(3-chloroallyl)-3,5,7-triaza-l-azonia-adamantan-chloride or the ammonium compound taurolidine.

Substances with antimicrobial action are usually contained in the ointment base (c2) in a quantity of 0 to 15 weight per cent, preferably from 0.1 to 5 weight per cent relative to the total weight of the ointment base.

The open-cell foam (c1) is soaked with the ointment base (c2). In this context “soaked” is taken to mean that the ointment base covers the outer and inner surfaces (=the inner surface formed by the open pores) of the foam, preferably fully. In a preferred embodiment at least 20%, more preferably at least 50%, even more preferably at least 70%, in particular at least 90% of the total surface area of the foam (c1) is covered with ointment base. The soaking of the surface of the foam with ointment base can also be referred to as impregnation.

It has been demonstrated that the objects cited at the beginning can be solved especially advantageously by using a specific quantity of ointment base. In a preferred embodiment the proportion of ointment base (c2) is 10 to 95 weight per cent, more preferably 30 to 92 weight per cent, even more preferably 45 to 90 weight per cent, especially preferably 55 to 88 weight per cent, in particular 65 to 85 weight per cent relative to the total weight of the wound dressing (c). The total weight of the wound dressing (c) is the sum of the weights of the components (c1) and (c2).

In principle, the explanations of preferred embodiments of individual parameters of the polyurethane foam (c1) and/or the ointment base (c2) must not be seen in isolation, but in combination with the explanations of preferred embodiments of other parameters or in combination with the explanations of the substance compositions of (c1) and (c2).

The wound dressing containing (c1) and (c2) can be advantageously produced using the method in accordance with the present invention. This includes the steps of:

(I) heating an ointment base (c 1) above the dropping point;

(II) introducing an open-cell foam (c2) into the heated ointment base so that the foam is soaked with ointment base,

(III) as applicable, temporarily compressing the foam, preferably to at least 50% and at the most 90% of its original volume in order to achieve the full soaking of the inner surface of the foam with the ointment base, and

(IV) as applicable, removing the surplus ointment base, preferably by squeezing the foam.

In step (I) the ointment base is heated up, preferably to around 10 to 30° C. above the dropping point.

In step (II) the foam is introduced and preferably immersed. The length of time immersed in the ointment base depends on the size of the foam and is usually 10 to 100 seconds.

Step (III) can be carried out during or after, preferably during immersion.

“Temporary compression” of the foam is taken here to mean that the foam is squeezed manually or by a suitable machine and then released. The foam can be released immediately after compression or held compressed for a brief period, for example for 1 to 10 seconds, and then released. After the foam is released following temporary compression, the foam can return fully or largely to its original volume. During the relaxation process ointment is usually absorbed, thereby fully soaking the inner surface of the foam with the ointment base.

Then the foam soaked with ointment base is removed. Surplus ointment base can be squeezed out (IV). This preferably takes place before the ointment base has cooled below its dropping point. The compression force is preferably selected in such a way that the proportion of ointment base is 10 to 95 weight per cent, more preferably 30 to 92 weight per cent, even more preferably 45 to 90 weight per cent, especially preferably 55 to 88 weight per cent, in particular 65 to 85 weight per cent relative to the total weight of the wound dressing.

All of the above explanations of preferred embodiments of the components (c), (c1) and (c2) also apply to the method in accordance with the present invention.

Furthermore, the invention provides a ready-to-use set for negative pressure wound therapy, including the device in accordance with the present invention, whereby the foam (c1) is suitable as a wound dressing and is provided in a ready-to-use pack.

The object of the invention is thus a ready-to-use set for negative pressure wound therapy comprising

(a) a cover material for air-tight sealing of the wound space, i.e. the wound and the area surrounding the wound,

(b) as applicable, a means suitable for the connection of a negative pressure source, preferably a means for the functional connection of the wound space with a negative pressure source outside of the cover material in such a way that a negative pressure can be generated in the wound space and fluids can be drawn out of the wound space by suction, and

(c) a wound dressing in a ready-to-use pack, comprising

(c1) an open-cell foam which is

(c2) soaked with an ointment base which preferably has a dropping point of 20 to 80° C. Preferably the proportion of the ointment base is 10 to 95 weight per cent relative to the total weight of the wound dressing. Preferably the ointment base is a triglyceride ointment base.

The wound dressing (c) included in the set as a ready-to-use pack should preferably be provided in a damp-proof pack. Preferably the ready-to-use wound dressing is provided in sterile form, whereby especially radiation can be used for sterilization. The set can contain further optional elements such as adhesive means to fix the dressing, sealing means to generate an air-tight seal of the dressing, pressure sensors, connection elements for pressure sensors, additional tubes, connection pieces for tubes, disinfectants, skin care products, pharmaceutical preparations or instructions for use. The set in accordance with the present invention preferably also contains scissors, pads and/or pincers, in particular in sterile form.

The set can include both the at least one wound contact layer as well as at least one additional pressure distribution layer. Preferably the set also contains a ready-to-use negative pressure unit.

A further object of the invention is the use of the wound dressing (c) explained above for or in the negative pressure wound therapy. An object of the invention is thus also the use of an open-cell foam (c1), which is soaked with an ointment base (a), for the negative pressure therapy of wounds, in particular as a wound dressing (c). All of the above explanations of preferred embodiments of the components (c), (c1) and/or (c2) also apply individually or in combination to the method in accordance with the present invention

The invention thus relates, for example, to the use of a polyurethane foam (c 1), which is obtainable by reaction of a mixture including the components

(i) polyisocyanate,

(ii) polyol, in particular polyester polyol,

(iii) blowing agent, and

(iv) catalyst;

whereby the foam is preferably obtainable by reaction of a polyisocyanate (i), selected from MDI, PMDI and/or TDI, with a (ii) polyester polyol, which is preferably obtainable by reaction of a dicarboxylic acid with 4 to 8 carbon atoms with a dialcohol with 2 to 6 carbon atoms, whereby the (ii) polyester polyol preferably has a weight average molecular weight of 500 to 4,000 g/mol;
whereby the open-cell polyurethane foam preferably has a proportion of aromatic compounds of 5 to 50%, more preferably from 10 to 45%, in particular from 15 to 40%;
whereby the open-cell polyurethane foam preferably has a ductile yield of 150% to 700%, more preferably from 200% to 650%, even more preferably from 240% to 400%, in particular 260% to 320%;
whereby the open-cell polyurethane foam preferably has a mechanical loss factor of 0.1 to 1.0, more preferably from 0.15 to 0.8, even more preferably from 0.2 to 0.6;
whereby the open-cell polyurethane foam preferably has a hardness of 20 to 70 Shore A, more preferably from 30 to 60 Shore A, even more preferably from 40 to 50 Shore A;
whereby the open-cell polyurethane foam preferably has a cell number (=number of pores along a straight line per cm) from 3 to 40 cm⁻¹, preferably from 5 to 25 cm⁻¹, more preferably from 7 to 18 cm⁻¹, even more preferably from 8 to 15 cm⁻¹;
whereby the open-cell polyurethane foam preferably has a raw density between 15 and 55 kg/m³, more preferably between 20 and 45 kg/m³, even more preferably between 22 and 38 kg/m³, in particular between 23 and 35 kg/m³; and/or
whereby the open-cell polyurethane foam preferably has an air permeability of 1,000 to 8,000 1/(m²sec), more preferably from 1,500 to 6,000 1/(m²sec), even more preferably from 2,000 to 5,000 l/ (m²sec) and especially preferably from 2,300 to 4,000 l/(m²sec) 2,400 to 3,300 l/(m²sec). In this use in accordance with the present invention, this polyurethane foam (c1) is soaked with an ointment base (c2) which has a dropping point of 20 to 80° C., preferably from 25 to 55° C. more preferably from 30 to 50° C., even more preferably from 33 to 48° C. and in particular from 35 to 45° C.;

which is preferably selected from a hydrophobic, water-absorbent and/or hydrophilic ointment base;

whereby the ointment base (c2) preferably contains triglycerides and in particular a mixture of triglycerides and diglycerides, e.g. 25 to 90 weight per cent, preferably 45 to 80 weight per cent triglycerides, and 10 to 75 weight per cent, preferably 20 to 55 weight per cent diglycerides.

Special advantages of the device in accordance with the present invention, the set in accordance with the present invention or the use or application in accordance with the present invention, arise when the wounds are burn wounds, wounds caused by mechanical trauma, chronic wounds caused by exposition to chemicals, chronic wounds caused by a metabolic disorder, chronic wounds caused by a circulatory disorder or wounds caused by pressure ulcers.

In a further preferred embodiment, the wound dressing (c) (including (c1) and (c2)) is provided for use in negative pressure therapy in the treatment of a wound caused by a skin graft. The application includes the treatment of wounds caused by split-skin and full-skin transplants using negative pressure therapy. Advantageous effects arise due to the structure of the open-cell foam (c 1) soaked with the ointment base (c2) and due to the uniform distribution of pressure. When the wound dressing (c) is used in the treatment of a wound caused by a skin graft, the skin graft can be adequately fixed while avoiding undesired shear forces.

The wound dressing (c) described above can be used advantageously as a wound dressing in the negative pressure therapy of pressure wounds in patients with a body-mass index (BMI=body weight over height squared) of less than 18.0, in particular with a body mass index of 14 to 17.5. This applies in particular to patients aged over 60. The advantageous effect of the device in accordance with the present invention or the set in accordance with the present invention is manifested in particular in such patients.

Another object of the invention is a method for negative pressure wound therapy. comprising the steps of

a) providing a device according to one of claims 1 to 10;
b) applying the negative pressure dressing to the wound;
c) generating a negative pressure of 25 mm Hg to 250 mm Hg, preferably 50 mm Hg to 150 mm Hg in the wound space for at least 30 minutes and up to a maximum of 7 days. preferably for at least 1 day and up to a maximum of 6 days.

### FIGURES

FIG. 1: Schematic view of the device in accordance with the present invention (side view)

FIG. 2: Apparatus for determination of the dropping point

FIG. 3: Pressure data obtained from the wound simulator in accordance with Example 2

### FIG. 1-Legend:

1 Wound surroundings (i.e. generally undamaged skin)

2 Air-tight cover material (a)

3 Wound dressing (c) =open-cell foam (c), soaked with Ointment base (c2)

4 Negative pressure connector (port)

5 Negative pressure connection line

6 Collector

7 Negative pressure unit

8 Wound

The device in accordance with the present invention for negative pressure wound therapy is explained in more detail in FIG. 1. The invention should not be seen, however, as being restricted to the embodiments shown in the drawings or in the description of the drawings. Rather, the device in accordance with the present invention also includes combinations of the individual features of the alternative forms.

FIG. 1 shows a schematic view (side view) of the device in accordance with the present invention. The device comprises an air-tight cover material (2), a means (4-5) for the functional connection of the wound space with a negative pressure source (7) outside of the cover material, and the open-cell foam (3). The cover material (2) is fastened in the area of the wound surroundings (1), usually consisting of undamaged skin. The size of the cover material must be such that the cover material can be fastened outside of the wound space in the area of the wound surroundings (1). The cover material (2) can have different dimensions and shapes, for example circular, oval or rectangular. It can also have an irregular shape matched to the individual wound. The cover material (2) is usually fastened in the area of the wound surroundings (1) and sealed air-tight. This can be achieved, for example, by providing an adhesive edge on the cover material (2). Alternatively an adhesive substance can be applied either to the edge of the cover material (2) and/or the intact skin around the wound. This has the advantage that it is easier to match the cover material to the shape and size of the wound. In the preferred embodiment shown here, the negative pressure connector (4) is attached to the outside of the air-tight cover material (2) facing away from the wound. In order to functionally connect the wound space with a negative pressure unit (7) outside of the cover material in this arrangement, there must be one or more openings passing through the cover material (2) in the proximity of the negative pressure connector (4).

In addition to this, an air-tight seal has to be ensured. Such a seal can be achieved. for example, if a foil (not shown in FIG. 1) is applied on the upper side of the port facing away from the wound which is adhered to the cover material (2). The application of the dressing is made easier if a port is used which already has a suitable means of fastening and sealing to fasten the port to the cover material. This is the case, for example, in the commercially available PPM-Drainageport® by the company Phametra-Pharma nod Medica-Trading GmbH (Herne/Ruhrstadt, Germany).

In a preferred embodiment of the invention the device for the negative pressure wound therapy includes no wound contact layer between the wound dressing (3) and the wound surface (8).

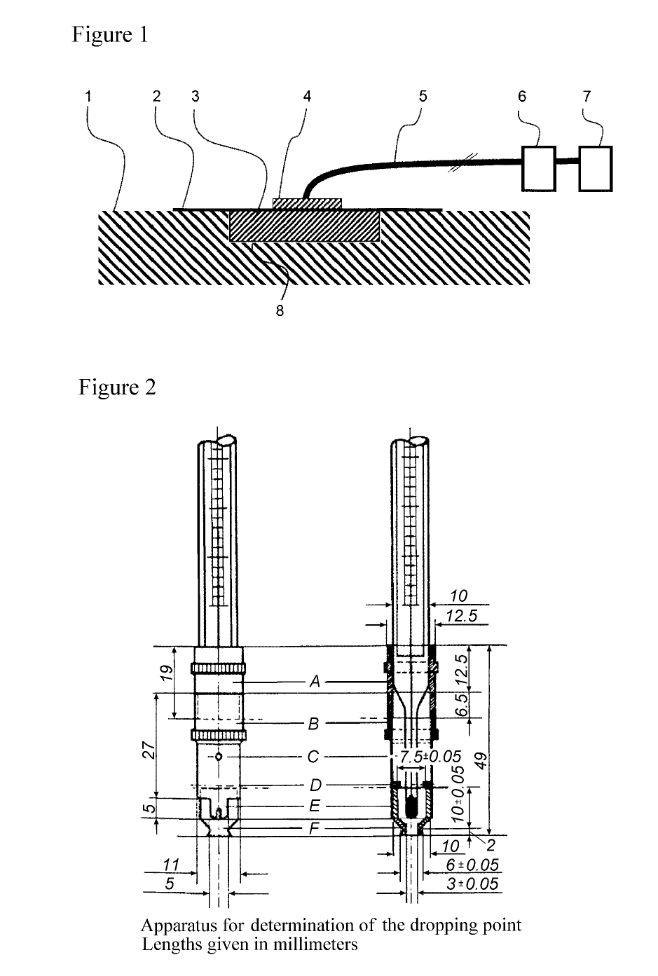

FIG. 2 shows the apparatus for determination of the dropping point. This determination is carried out experimentally as follows:

Test apparatus: dropping point thermometer according to Ubbelohde

0-110° C., calibrated

glass beaker 1,000 ml

test tube, ca. 200 mm long, diameter: ≈40 mm, with holed stopper

magnetic stirrer with hotplate

filter paper 10×10 mm

Reagents: demin. water

apparatus for determination of the dropping point in accordance with FIG. 2

The apparatus (see FIG. 2) is made up of two metal sleeves (A) and (B) screwed together. The sleeve (A) is fastened to a mercury thermometer. A metal nipple (F) is loosely attached to the lower end of sleeve (B) with 2 clamps (E). 2-mm locking pins (D) fix the nipple in the exact position. They also serve to center the thermometer. An opening (C) in the wall of the sleeve (B) serves to relieve pressure.

The drip surface of the nipple must be flat and the edges of the outlet opening must be perpendicular to this. The lower part of the mercury thermometer has the shape and dimensions as shown in the figure. The thermometer allows temperature measurement of 0 to 110° C. in increments of 1° C. (1 mm each). The mercury vessel of the thermometer has a diameter of 3.5±0.2 mm and a height of 6.0±0.3 mm. The whole apparatus was suspended at the center of a ca. 200-mm test tube of 40 mm outer diameter with the aid of a holed stopper through which the thermometer was inserted. The stopper has a notch at the side. The nipple opening must be 15 mm over the bottom of the test tube. The whole apparatus was immersed in a 1-liter glass beaker filled with water. The bottom of the test tube must be around 25 mm above the bottom of the beaker. The water level must reach the upper part of the sleeve (A). A stirrer ensures a uniform temperature of the water bath.

The test method was carried out as follows:

The nipple was filled completely with the unmelted substance to be tested insofar as nothing else is prescribed. The surplus substance is scraped off both ends of the nipple using a spatula. The sleeves (A) and (B) were screwed together and the nipple inserted into the sleeve (B) up to the locking pins. The substance pressed out by the thermometer at the nipple opening was scraped off with a spatula. The apparatus was suspended in the water as described above. Then the water was heated up in such a way that from around 10° C. below the expected dropping point the temperature was increased by around 1° C. per minute. The temperature was read off when the first drip fell from the nipple. The experiment was carried out at least three times with new samples whereby the individual values could only deviate from each other by a maximum of 3° C.

Evaluation: The dropping point is the mean value of the three determinations. The invention is explained in the following examples.

### EXAMPLES

### Example 1

Manufacture of the wound dressing (c) in accordance with the present invention

An ointment base containing a triglyceride/diglyceride mixture (dropping point ca. 40° C.) was heated up to 55° C. An open-cell polyester polyol-polyurethane foam (raw density 28 kg/m³, thickness 32 mm) was immersed and compressed to around 70% of its original volume so that when the foam returned to its original volume after being compressed, the inner surface of the foam was fully soaked with ointment base. After it was taken out, the foam was squeezed out so that the proportion of ointment base after squeezing was 79 weight per cent.

### Example 2

Testing the wound dressing in accordance with Example 1 in the wound simulator

The wound dressing in accordance with Example 1 was tested in the negative pressure wound simulator (described in DE 10 2008 064 510).

For a “successful” simulation of a negative pressure therapy, the results of the test only had to show very low pressure differences between the wound space (sensor 1) and the port area (sensor 2) as well as uniform behavior over a prolonged period with simultaneous linearity in terms of the exudates withdrawn. Any difference in pressure is due to the wound dressing. The negative pressure was generated using an ATMOS SO41 wound drainage pump. The exudate was generated by a B. Braun Perfusor F® spray pump which can generate a constant flow. A PPM drainage port system (Herne/Ruhrstadt, Germany) was used.

TABLE 1
Parameters

| | | | Exudate | | |
|---|---|---|---|---|---|
| | Temper- | Pres- | Flow | Viscosity | Wound |
| | ature | sure | (ml · | (mPa · s) | size |
| Wound dressing | (° C.) | (mm Hg) | h⁻¹) | (albumin) | (cm²) |
| Wound dressing | 32 | 125 | 2 | 6.4 | 28.3 |
| in accordance | | | | | |
| with the present | | | | | |
| invention as in | | | | | |
| Example 1 | | | | | |

The wound dressing was placed on the wound simulator and covered with Hydrofilm® to ensure an air-tight system. Over the artificial wound a small hole was cut into the film and a PPM port was applied to allow the exudate to flow out of the wound space. This port was connected with a container to collect the exudate, and with the pressure gauge to measure the pressure within the port system. The container was connected with a vacuum pump and the quantity of the extracted exudate measured by determining the weight of the exudate on a scale. The other pressure sensor, which was fitted inside the wound simulator, measured the pressure within the simulated wound. The trial was carried out using the parameters shown in Table 1.

### Data Analysis

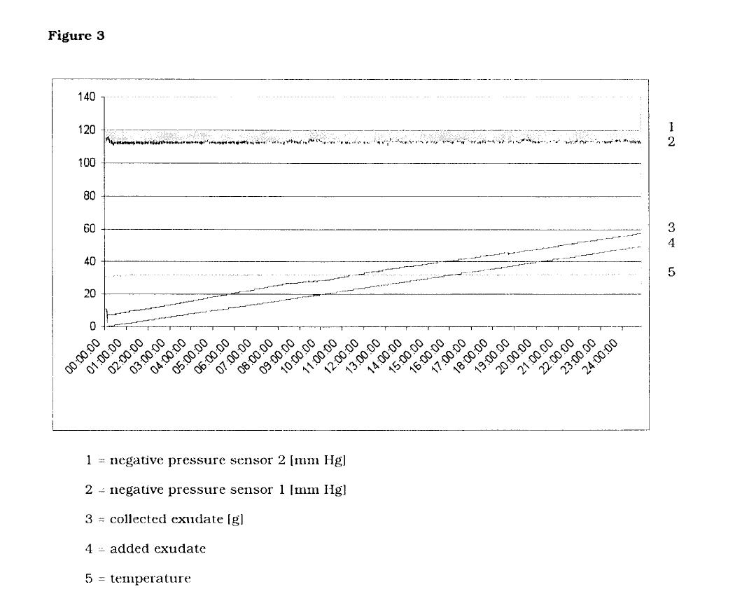

The trial was carried out over a period of 24 hours and 53 minutes. The results received for the two pressures cited above (FIG. 3) are given in the following. Sensor I measured the pressure inside the wound, while sensor 2 determined the pressure in the port system. Mean values were calculated for the respective pressures and the pressure differences (Table 2).

TABLE 2
Sensor 1 mean value (mmHg):
113.69
Sensor 2 mean value (mmHg):
115.90
Pressure difference mean value (mmHg):
2.21

The results show that the pressure difference remained low and the behavior of the pressures normal during the whole trial. The calculated and collected exudates show a very similar graph, thereby proving the success of the trial.

This leads to the following conclusion.

The quantity of the collected exudate increased linearly as expected and the pressure difference showed very good results. This means that the wound dressing in accordance with the present invention as in Example 1 functioned consistently and unexpectedly advantageously in an NPWT simulation trial.

1. A device for negative pressure wound therapy comprising
(a) a cover material for air-tight sealing of the wound space;
(b) as applicable, a connector of a negative pressure source; and
(c) a wound dressing comprising
(c1) an open-cell foam which is
(c2) soaked with an ointment base which has a dropping point of 20 to 80° C., whereby the proportion of ointment base is 10 to 95 weight per cent relative to the total weight of the wound dressing.

2. The device in accordance with claim 1, whereby the ointment base (c2) contains a triglyceride.

3. The device in accordance with claim 2, whereby the triglyceride used in the ointment base (c2) contains a glycerin residue and three C₆—C₂₈ acid residues, preferably C₈—C₁₈ acid residues.

4. The device in accordance with claim 1, whereby the ointment base (c2) contains a diglyceride.

5. The device in accordance with claim 4, whereby the diglyceride used in the ointment base (c2) contains a glycerin residue and two C₄—C₂₈ acid residues, preferably C₆—C₁₈ acid residues.

6. The device in accordance with claim 1, whereby the foam (c1) is a polyurethane foam which can be obtained by reaction of a mixture comprising the components
(i) polyisocyanate,
(ii) polyol, in particular polyester polyol,
(iii) blowing agent, and
(iv) catalyst.

7. The device in accordance with claim 1, whereby the foam (c1) has a tensile strength of 100 kPa to 500 kPa, measured in accordance with DIN 53571 and/or a ductile yield of 200% to 700%, measured in accordance with DIN 53571.

8. The device in accordance with claim 1, whereby the foam (c1) has an air permeability of 1,000 to 8,000 1/(m²sec), measured in accordance with DIN EN ISO 9237.

9. The device in accordance with claim 1, whereby the foam (c1) has a raw density between 15 and 40 kg/m³, measured in accordance with DIN EN ISO 845.

10. The device in accordance with claim 1, whereby the cover material (a) has a water vapor permeability of 100 to 2,500 g/m²×24 h, measured in accordance with DIN EN 13726-2.

11. A method for the manufacture of a wound dressing comprising the steps of
(I) heating an ointment base above the dropping point,
(II) introducing an open-cell foam into the heated ointment base, so that the foam is soaked with the ointment base,
(III) as applicable, temporarily compressing the foam, preferably to at least 50% and up to a maximum of 90% of its original volume in order to ensure that the internal surface of the foam is fully soaked with the ointment base, and
(IV) as applicable, removing the surplus ointment base, preferably by squeezing the foam.

12. The method in accordance with claim 11, whereby the compression force is selected in such a way that the proportion of ointment base is 10 to 95 weight per cent relative to the total weight of the wound dressing.

13. A wound dressing obtainable by a method in accordance with claim 11.

14. (canceled)

15. (canceled)

16. A method for negative pressure wound therapy, said method comprising dressing a wound with an open-cell foam soaked with a triglyceride ointment base, and applying negative pressure to the wound.

17. The method of claim 16, wherein the wound is a burn wound, a wound caused by mechanical trauma, a wound caused by exposure to chemicals, a wound caused by a metabolic disorder, a wound caused by a circulatory disorder, or a wound caused by pressure ulcer.

18. The method of claim 16, wherein the wound dressing is manufactured by a method comprising the steps of
(I) heating an ointment base above the dropping point,
(II) introducing an open-cell foam into the heated ointment base, so that the foam is soaked with the ointment base,
(III) as applicable, temporarily compressing the foam, preferably to at least 50% and up to a maximum of 90% of its original volume in order to ensure that the internal surface of the foam is fully soaked with the ointment base, and
(IV) as applicable, removing the surplus ointment base, preferably by squeezing the foam.
